(19) United States
(12) Patent Application Publication
(10) Pub. No.: US 2025/0380862 A1
(43) Pub. Date: Dec. 18, 2025

(54) VIBRATING BRUSH FOR ENDOSCOPE REPROCESSING

(71) Applicant: Boston Scientific Scimed, Inc., Maple Grove, MN (US)

(72) Inventors: Dabne Celin Barrera Trujillo, Bogotá (CO); Catalina Tirado Aldana, Bogotá (CO); Santiago Sanchez Renteria, Bogotá D.C (CO); Carolina Villarreal, Hopedale, MA (US); Daniela Cespedes Valenzuela, Bogotá (CO)

(73) Assignee: Boston Scientific Scimed, Inc., Maple Grove, MN (US)

(21) Appl. No.: 19/233,929

(22) Filed: Jun. 10, 2025

Related U.S. Application Data
(60) Provisional application No. 63/659,185, filed on Jun. 12, 2024.

(51) Int. Cl. A61B 1/12; A46B 5/00; A46B 5/06; A46B 9/02; A46B 13/02; A46B 15/00; A46D 1/00

(52) U.S. Cl. CPC A61B 1/122; A46B 5/0012; A46B 5/06; A46B 9/025; A46B 13/023; A46B 15/0004; A46D 1/0207; A46B 2200/3013

(57) ABSTRACT

Devices, systems, and methods for cleaning endoscopic channels. A brush may have multiple brush heads along a wire and a vibration device. The vibration device may have motor-controlled gears to hold the wire and move the wire and brush heads with both forward and backward movements to clean the channel.

### CROSS-REFERENCE TO RELATED APPLICATIONS

This application claims the benefit of U.S. Provisional Patent Application Ser. No. 63/659,185 filed on Jun. 12, 2024, the disclosure of which is incorporated herein by reference.

### FIELD

This disclosure relates generally to cleaning devices and methods, and particularly for cleaning devices for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages.

Because of the high costs associated with manufacturing these components, many are designed to be reusable. However, the reuse of endoscopic medical devices is in tension with the need for a sterile medical environment. There is therefore a need for efficient and effective procedures to thoroughly clean and sanitize endoscopic devices prior to reuse.

### SUMMARY

This disclosure provides design, material, manufacturing method, and use alternatives for medical devices and medical systems. In a first example, a brush for cleaning an endoscopic medical device includes a wire; a plurality of brush heads disposed along the wire; and a vibration device. The vibration device has a plurality of gears configured to receive the wire; one or more motors configured to drive the plurality of gears; and a controller configured to control the one or more motors. The controller has a non-transitory computer-readable medium having instructions that, when executed by the controller, cause the wire and brush heads to be moved in a pattern of both forward and backward movements to clean an endoscopic channel.

Alternatively or additionally to any of the examples above, the plurality of brush heads can include at least one brush head having bristles tilted forward and at least one brush head having bristles tilted backward.

Alternatively or additionally to any of the examples above, the plurality of brush heads can include a front and rear brush head having bristles tilted forward and a middle brush head having bristles tilted backward.

Alternatively or additionally to any of the examples above, the brush heads can have bristles made of nylon or plastic.

Alternatively or additionally to any of the examples above, the one or more motors can be a single motor configured to drive the gears in tandem.

Alternatively or additionally to any of the examples above, the one or more motors can be a different motor driving each gear of the plurality of gears.

Alternatively or additionally to any of the examples above, the pattern of forward and backward movements can include a forward movement of a first length alternating with a backward movement of a second length, the first length being longer than the second length.

Alternatively or additionally to any of the examples above, the first length can be twice the second length.

Alternatively or additionally to any of the examples above, the one or more motors can be one or more stepper motors.

Alternatively or additionally to any of the examples above, the vibration device can include a housing.

Alternatively or additionally to any of the examples above, the housing can include a compartment to receive a disposable cartridge. The wire can be coiled within the cartridge for dispensing by the vibration device.

Alternatively or additionally to any of the examples above, the one or more motors and the controller can be contained within the housing. The gears can hold and move the wire outside of the housing.

Alternatively or additionally to any of the examples above, the brush can also include a user interface for initiating and halting the operation of the vibration device.

Alternatively or additionally to any of the examples above, the wire and brush heads can be disposable after a single use.

In another example, a method of cleaning an endoscopic channel includes inserting a brush of any of the above claims into the endoscopic channel; and executing, by the controller, the instructions to cause the wire and brush heads to be moved in a pattern of both forward and backward movements to clean the endoscopic channel.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3 is a flowchart illustrating an exemplary medical device cleaning process;

FIG. 4 depicts a schematic view of an illustrative brush;

FIG. 5 depicts a diagrammatic view of the components of an illustrative brush;

FIG. 6 depicts a cross-sectional plan view of an illustrative brush; and

FIG. 7 depicts a perspective view of a vibration device of an illustrative brush.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

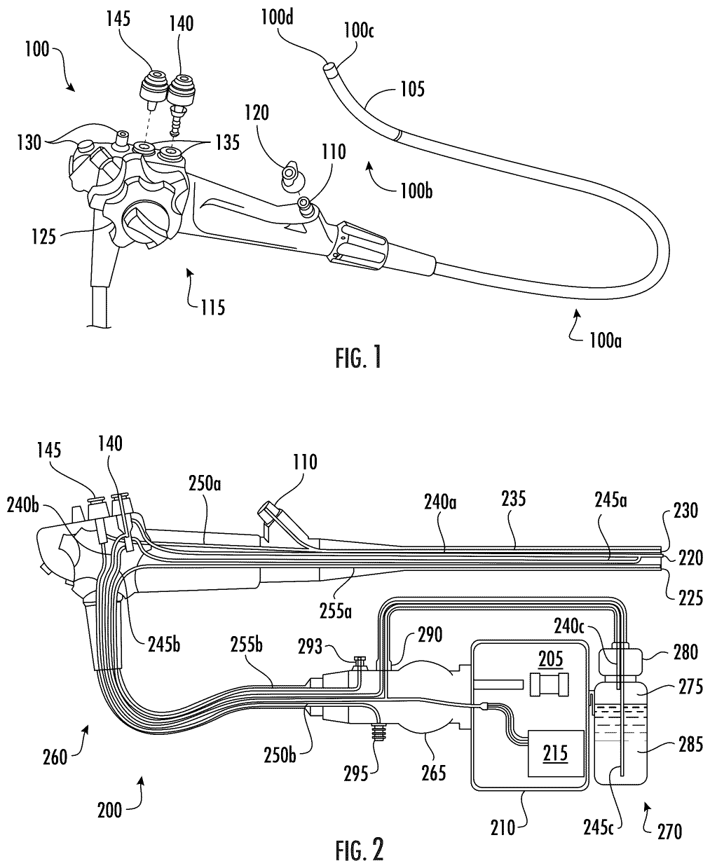

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serves as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

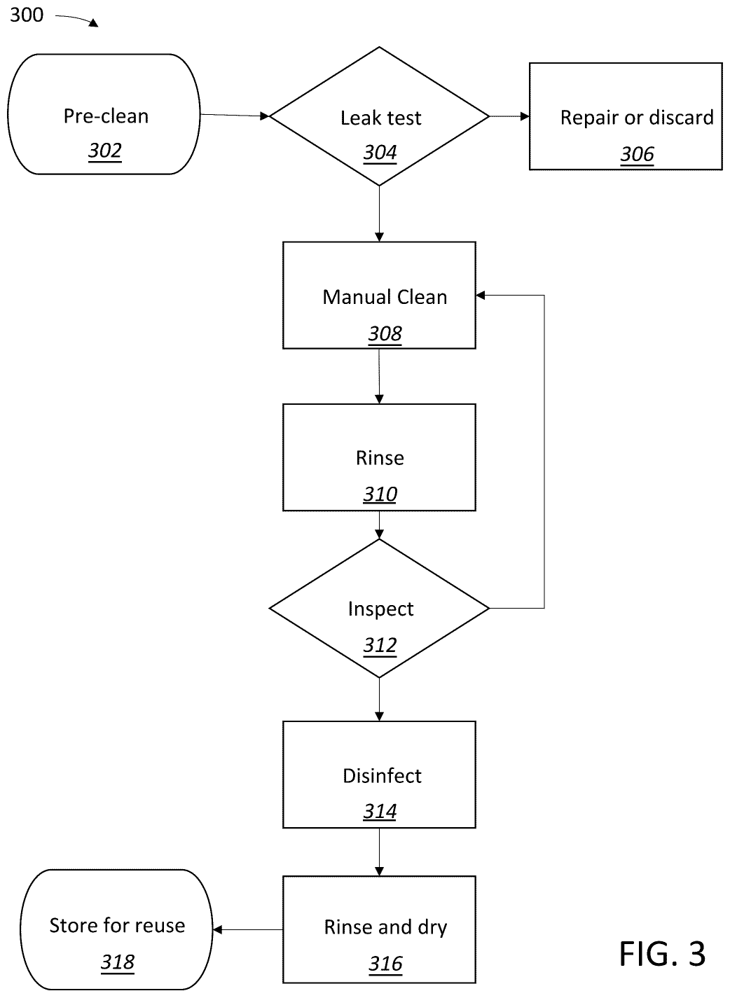

After the use of the above system 200 in an endoscopic procedure, some of the components may be reprocessed for subsequent reuse. An example of such a reprocessing method 300 is shown in FIG. 3.

In step 302, components of the system are subject to initial cleaning. This pre-cleaning step may be carried immediately after the endoscopic procedure or at some later time. Pre-cleaning may involve an initial rinse and/or immersion to rid components of any visible external fluid or detritus. In some implementations, pre-cleaning may be performed at a cleaner's discretion based on informal or casual inspection of the components to be cleaned.

Following pre-cleaning, in step 304, leak testing may be performed on one or more of the components, particularly the tubing. This may involve running a sterile liquid, such as water or saline solution, through the supply and/or working channels to ensure that none escapes. Should any component be found to have a leak, it may be set aside for disposal or repair (step 306), and only components that pass the leak testing complete the remaining steps of the reprocessing as described.

A manual cleaning is performed at step 308. Here, brushes and other tools are used to remove any debris. It is during this manual cleaning step that, in some implementations, a cleaning brush of the sort described herein may be used. The handle 115 and any of the tubing described herein may be cleaned. Particularly the working channel 235 and any other tubing that exposed to biological material such as patient fluids, samples, or waste may be included in the manual cleaning. For each channel to be cleaned, cleaning tools such as the cleaning brush are inserted and pushed entirely through the channel to capture and remove the debris in that channel.

One or more rinsing cycles follow the manual cleaning at step 310, which may involve any appropriate cleaning liquid such as distilled water, saline, detergent solution, or the like. A visual inspection is then performed (step 312) to confirm the efficacy of the cleaning and rinsing steps; a failed inspection may then require more manual cleaning (returning to step 308).

Should the inspection confirm that the component or components are satisfactorily free of debris, the devices may then be disinfected at step 314. The disinfecting process may involve the exposure of components to radiation, temperature, liquids, gases, or any combination of these, and may be carried out over any time period sufficient to thoroughly clean the device to the standards necessary for medical reuse. In some implementations, the disinfection process may meet regulatory and industry standards for High-Level Disinfection.

Following the disinfection process, the components are again rinsed and dried to remove any residue (step 316) prior for being stored for reuse (step 318).

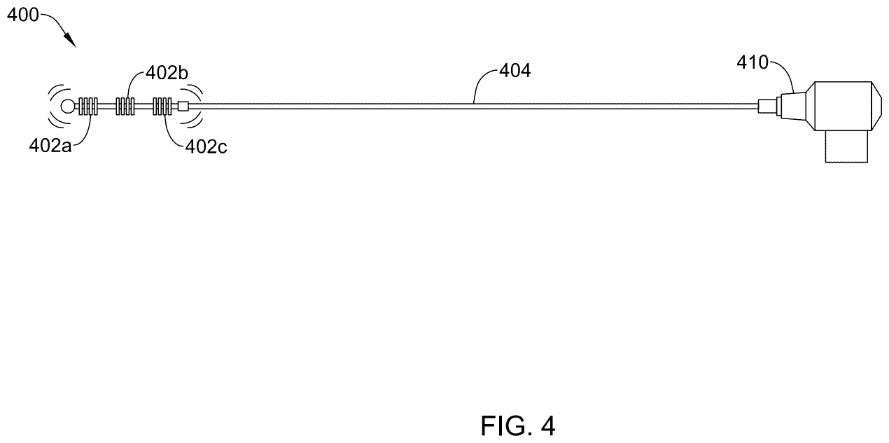

A vibrational brush device for use in the manual cleaning step is shown in FIG. 4. The brush 400 comprises three bristle heads 402a-c disposed along a wire 404. The bristle heads may be oriented in opposite directions to facilitate more efficient cleaning of the endoscopic channel. For example, the front bristle head 402a and the rear bristle head 402c may have bristles that are tilted toward the front of the brush, while the middle bristle head 402b is tilted toward the rear of the brush. The bristles of each bristle head may be made of a semi-rigid material appropriate for the manual cleaning process and the endoscopic channel, such as nylon or thermoplastic. The diameter of the brush heads is selected according to the size of the endoscopic channel such that the brush will contact the

The bristle heads are moved within the endoscopic channel by use of a vibration device 410. Gears 500 hold a portion of the wire 404 and move the brush both forward and backward in order to clean the endoscopic channel.

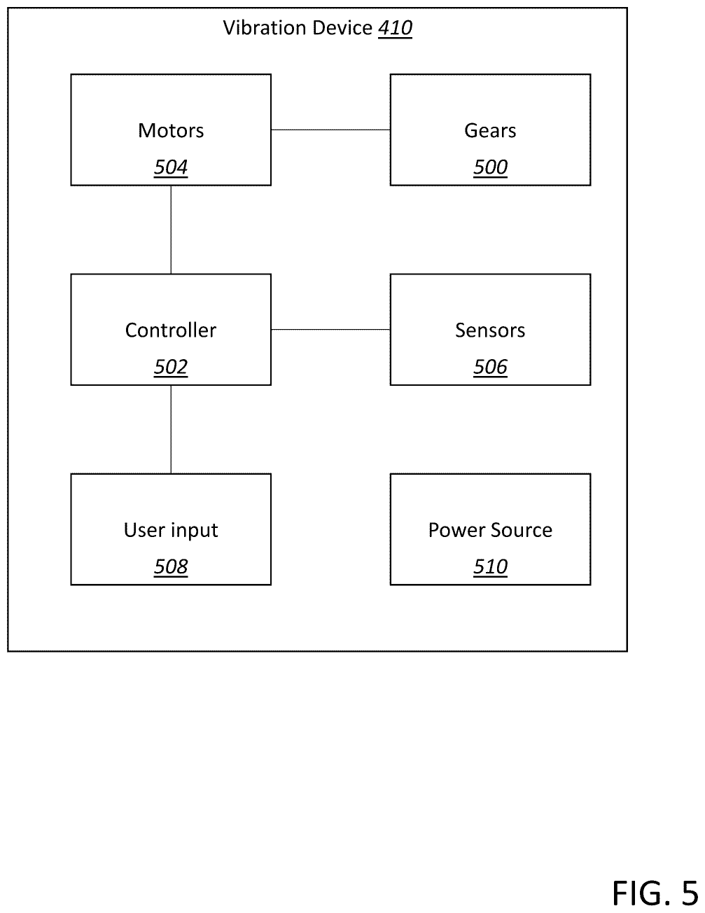

As diagrammed in FIG. 5, motion of the gears 500 comes from motors 504, which may be stepper motors, gear motors, or the like. In some implementations, the motors 504 may instead be a single motor configured to move the gears 500 in tandem. The motors 504 receive direction from a controller 502, which in turn may include any or all of the components of a microcontroller module, such as digital logic, memory, a processor, and the like. One of ordinary skill will recognize variations in both the controller 502 and motors 504 to carry out the functions described herein.

The vibration device 410 may further include one or more sensors 506 providing data to the controller 502. Sensors 506 may include temperature and moisture sensors to prevent device failure, data on motor and/or gear position to aid in operation, the location and status of various components (such as, for example, the presence and condition of a cartridge included in some implementations as described below), and the like. Some implementations may include few or no sensors.

The vibration device 410 may include any form of user input 508 to allow the user to instruct the controller 502. In some implementations, the user input 508 may be an on/off button that, when pressed, engages a preset cleaning process or halts the process. The user input 508 may include additional switches, buttons, dials, and touch-sensitive surfaces to select parameters for cleaning. In some implementations, a user may be able to vary the speed, duration, and/or pattern of the brush movements by means of the user input 508.

The power source 510 may be common to the motors 504, controller 502, and other components of the vibration device 410. Portable power sources such as batteries may be used. Wall power, in the form of an electrical adapter, may be included as a continuous source of power. When used in a medical environment, the vibration device 410 may include one or more components allowing the device to work on emergency power and/or with power sources provided for medical devices.

The controller 502 may be programmed to move the brush 400 in a preset pattern that includes both forward and backward motion, but with a greater frequency and/or duration of forward motion such that the brush 400 advances through the endoscopic channel. For example, the controller 502 may advance a pattern of two cycles of forward motion (each of which may be some set number n of steps, for example, in a step motor) followed by one cycle of backward motion. The duration of each cycle and the ratio of cycles may vary depending on the brush configuration and endoscopic channel properties. Other patterns (such as three cycles forward, two cycles back, for example) are also possible. In some implementations, the patterns may vary during the course of the cleaning process. The controller 502 may be further programmed to vary the pattern based on sensed conditions of the brush and/or channel. The controller 502 may be pre-programmed to end motion after a set duration, to detect the end of the wire 404 and end motion, or to continue the programmed pattern until halted by input or other action from the user.

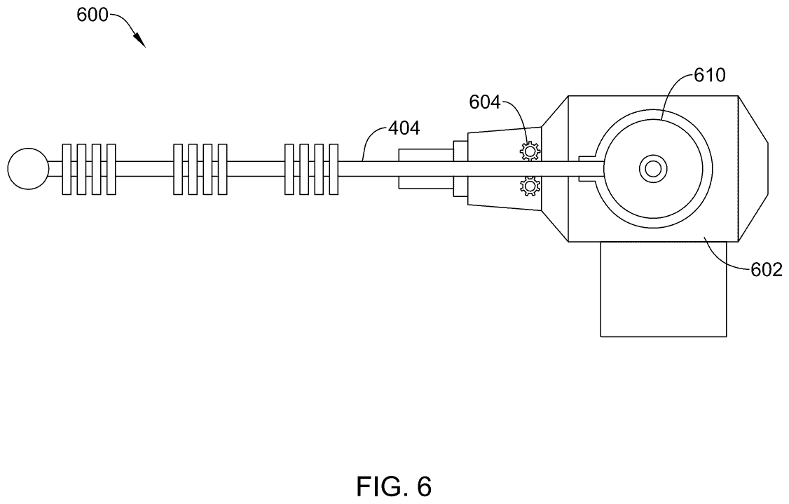

FIG. 6 shows a vibration device 600 generally similar to the device 410 described above. The vibration device 600 includes a housing 602 for the components described above including the motor-driven gears 604, and also with a compartment for a replaceable cartridge 610.

Each cartridge 610 holds a disposable brush of the sort described above as the brush 400. The wire 404 of the brush 400 is coiled within the cartridge 610, which is dispensed by the action of the gears as described above. Because the disposable cartridge 610 separates the brush from the components of the vibration device 600, the reusable device 600 is less susceptible to contamination during repeated use.

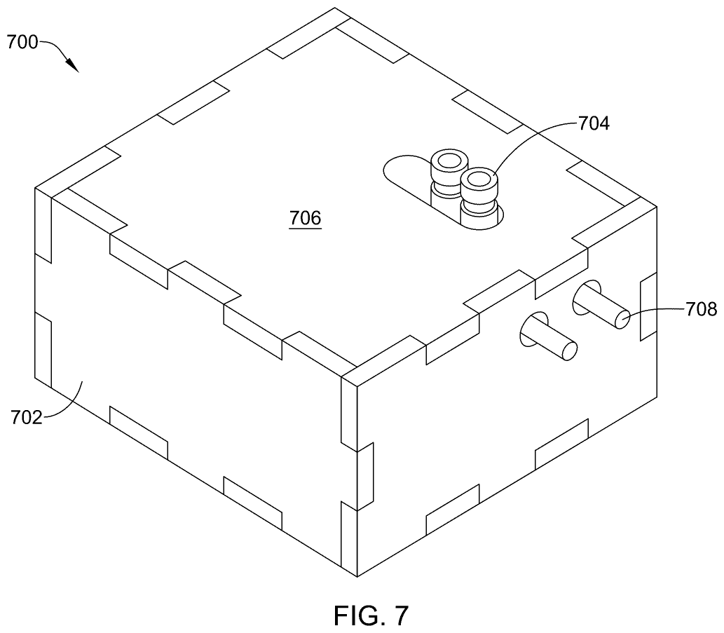

FIG. 7 shows another embodiment of a vibration device 700 in which the components are disposed within a casing 702, leaving only gears 704 and an outer surface 706 in direct contact with the endoscopic components and cleaning brushes. Clips, clamps, and other means may be used to hold the tubing in place while the brush is inserted into the endoscopic channel for cleaning. A user interface 708 on another surface of the casing 702 interacts with the controller to activate and deactivate the device 700.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A brush for cleaning an endoscopic medical device, comprising:
a wire;
a plurality of brush heads disposed along the wire; and
a vibration device, comprising:
a plurality of gears configured to receive the wire;
one or more motors configured to drive the plurality of gears; and
a controller configured to control the one or more motors, the controller comprising a non-transitory computer-readable medium having instructions that, when executed by the controller, cause the wire and brush heads to be moved in a pattern of both forward and backward movements to clean an endoscopic channel.

2. The brush of claim 1, wherein the plurality of brush heads comprises at least one brush head having bristles tilted forward and at least one brush head having bristles tilted backward.

3. The brush of claim 2, wherein the plurality of brush heads comprises a front and rear brush head having bristles tilted forward and a middle brush head having bristles tilted backward.

4. The brush of claim 1, wherein the brush heads have bristles made of nylon or plastic.

5. The brush of claim 1, wherein the one or more motors consists of a single motor configured to drive the gears in tandem.

6. The brush of claim 1, wherein the one or more motors consists of a different motor driving each gear of the plurality of gears.

7. The brush of claim 1, wherein the pattern of forward and backward movements comprises a forward movement of a first length alternating with a backward movement of a second length, the first length being longer than the second length.

8. The brush of claim 7, wherein the first length is twice the second length.

9. The brush of claim 1, wherein the one or more motors are one or more stepper motors.

10. The brush of claim 1, wherein the vibration device comprises a housing.

11. The brush of claim 10, wherein the housing comprises a compartment to receive a disposable cartridge, and wherein the wire is coiled within the cartridge for dispensing by the vibration device.

12. The brush of claim 1, wherein the one or more motors and the controller are contained within the housing, and wherein the gears hold and move the wire outside of the housing.

13. The brush of claim 1, further comprising a user interface for initiating and halting the operation of the vibration device.

14. The brush of claim 1, wherein the wire and brush heads are disposable after a single use.

15. A method of cleaning an endoscopic channel, comprising:
inserting a brush into the endoscopic channel, the brush comprising:
a wire;
a plurality of brush heads disposed along the wire; and
a vibration device, comprising:
a plurality of gears configured to receive the wire;
one or more motors configured to drive the plurality of gears; and
a controller comprising a non-transitory computer-readable medium having instructions; and
executing, by the controller, the instructions to control the one or more motors to move the wire and brush heads in a pattern of both forward and backward movements to clean the endoscopic channel.

16. The method of claim 15, wherein the plurality of brush heads of the brush comprises at least one brush head having bristles tilted forward and at least one brush head having bristles tilted backward.

17. The method of claim 15, wherein the pattern of forward and backward movements comprises a forward movement of a first length alternating with a backward movement of a second length, the first length being longer than the second length.

18. The method of claim 17, wherein the first length is twice the second length.

19. The method of claim 15, wherein the one or more motors of the brush are one or more stepper motors.

20. The method of claim 15, the brush further comprising a user interface in communication with the controller for initiating and halting the execution of the instructions.
